(19) United States
(12) Patent Application Publication
(10) Pub. No.: US 2021/0283320 A1
(43) Pub. Date: Sep. 16, 2021

(54) THORACIC DRAINAGE SYSTEM

(71) Applicant: REDAX S.P.A., POGGIO RUSCO (IT)

(72) Inventors: Lucio GIBERTONI, Mirandola (IT); Andrea GIBERTONI, San Possidonio (IT)

(21) Appl. No.: 16/467,672

(22) PCT Filed: Dec. 5, 2017
(86) PCT No.: PCT/EP2017/081500

§ 371 Date: Jun. 7, 2019

(30) Foreign Application Priority Data
Dec. 12, 2016 (IT) 102016000124793

(51) Int. Cl. A61M 1/04; A61M 27/00

(52) U.S. Cl. CPC A61M 1/04; A61M 25/0009; A61M 2210/101; A61M 27/00

(57) ABSTRACT

A thoracic drainage system having a drainage catheter that is adapted to be inserted in the pleural space of a patient in order to drain air and/or liquids that are present in the pleural space, and a drain tube, which is connected to the drainage catheter by a connector component. The drainage catheter includes longitudinally extended channels which are open radially toward the outside of the drainage catheter and at least one port for the drainage of the liquids, in which the open channels merge. The drainage catheter includes a port having at the distal end, a drainage catheter and radial holes. The drain tube has a first outflow port which is connected, by the connector component, to the at least one port and a second outflow port which is connected, by the connector component, to the port for the drainage of the air.

### TECHNICAL FIELD

The present disclosure relates to a thoracic drainage system for the drainage of air and/or liquids formed as a consequence of surgical procedures or traumas, from the thoracic cavity of a patient, in order to avoid their accumulation.

### BACKGROUND

In general, conventional thoracic drainage systems comprise a thoracic drainage catheter, i.e., a flexible tube that is provided by using transparent and sterile plastic materials, such as for example PVC (polyvinyl chloride) or silicone, and is inserted, through an incision, in the thorax of the patient until it reaches the pleural space in the thoracic cavity.

This drainage catheter constitutes the distal element of a thoracic drainage system and is therefore used to reach and remove the fluids, i.e., air and/or liquids, that have accumulated in the thoracic cavity.

In particular, usually air tends to accumulate in the apical region of the thoracic cavity of the patient, while liquids, such as for example blood, tend to accumulate in the basal region of the thoracic cavity of the patient.

Various types of drainage catheter for thoracic drainage systems are currently known. One of these thoracic drainage catheters of the known type is provided with a plurality of holes in its terminal portion, which is arranged in the apical region of the thoracic cavity of the patient, thus allowing the immediate outflow of the air toward the collection chamber, the liquids instead remaining deposited in the basal region of the thoracic cavity.

By using this known thoracic drainage catheter, the evacuation of the liquids, initially deposited in the basal region of the thoracic cavity, thus occurs when the lung, by expanding by virtue of the outflow of the air, pushes said liquids toward the apical region of said thoracic cavity, i.e., toward the holes that are present in the end portion of the drainage catheter and therefore toward the outlet of the catheter, up to the collection chamber.

It is evident that by using this thoracic drainage catheter of the known type, provided with holes in its terminal portion, the air and the liquids that are present in the thoracic cavity of the patient are evacuated alternately and not simultaneously, since the air exits first and the liquids exit only at a later time.

A different conventional thoracic drainage catheter has, as a replacement of the holes in the terminal portion, one or more grooves which are longitudinal with respect to the thoracic drainage catheter. This type of drainage catheter has been conceived in an attempt to allow the simultaneous evacuation of air and liquids from the thoracic cavity of the patient by using a single drainage catheter, but in this case the outflow of the liquids from the basal region of the thoracic cavity occurs immediately and the outflow of the air from the apical region of said thoracic cavity occurs only subsequently.

It is therefore evident that even by using this grooved thoracic drainage catheter of a known type the simultaneous outflow of the air and of the liquids that are present in the thoracic cavity of the patient does not occur, since the liquids exit first and the air exits only at a later time.

In view of the above, these currently known thoracic drainage catheters are not free from drawbacks, which include the fact that they do not allow the simultaneous and continuous evacuation of the air and of the liquids that are present in the thoracic cavity of the patient.

Another drawback of these conventional thoracic drainage catheters resides in that since they do not allow continuous evacuation of the air in the collection chamber, in practice they prevent medical staff from viewing immediately any air leaks of the patient.

One solution to the drawback described above, related to the evacuation from the thoracic cavity of the patient of air and liquids in two distinct and alternating steps, provides for positioning a pair of drainage catheters, both provided with a plurality of holes in their terminal portion, inside the thoracic cavity, one arranged in the apical region of the pleural space and the other one in the basal region.

The first drainage catheter, arranged in the apical region, allows the evacuation of the air, while the second drainage catheter, arranged in the basal region, allows the evacuation of the liquids. Both drainage catheters then merge in a three-way or Y-shaped connector and the fluids that they carry continue toward the collection chamber in a single tube.

Therefore, by using a pair of known thoracic drainage catheters the air and liquids that are present in the thoracic cavity of the patient can be evacuated simultaneously.

The drawback of these thoracic drainage catheters of the known type resides in that the only way to ensure simultaneous and continuous evacuation of the air and of the liquids that are present in the thoracic cavity of the patient is to use a pair of known drainage catheters, but this solution leads to a significant increase in the intensity of the pain felt by the patient, as well as to additional difficulties in the already complex management of the thoracic drainage system.

A further solution to the drawbacks described above provides for the use of a so-called coaxial drainage catheter, i.e., comprising two coaxial ports, wherein the internal port is assigned to the drainage of air while the external port is assigned to the drainage of liquids.

In particular, the catheter has, in the distal portion arranged to reside in the apical region of the pleural space, where air is present, a plurality of openings or windows which connect the internal port to the external space. In the proximal portion, arranged to reside in the basal region of the pleural space, where liquids are instead most present, the external space is connected only to the external port, by means of other openings or windows. In this manner the drainage catheter is capable of draining simultaneously and continuously both air and liquids through the two defined preferential paths.

However, even this last solution is not free from drawbacks.

In particular, the provision of drainage catheters provided with appropriate windows is rather complicated, time-consuming and expensive, since it requires a succession of different processes.

Furthermore, another drawback of drainage systems that provide for the use of the above-cited coaxial drainage catheters resides in that the air and the liquids are made to flow out together toward the collection chamber, which is often at a certain distance from the patient, through a drain tube.

If the drain tube becomes occluded, for example due to prolonged use of the thoracic drainage system, or due to the accumulation of liquids in a bend that has formed along the drain tube, air evacuation on the part of the patient is prevented. Indeed, if the patient who has air leaks to be evacuated is unable to exert such a positive pressure as to release the occlusion of the drain tube, severe respiratory and/or cardiac failures can arise, leading even to patient death.

One solution to the drawback cited above resides in applying to the drainage system, downstream of the drainage catheter, a separation filter, so as to separate the liquid phase from the gaseous phase and keep the two paths separate up to the collection chamber. In this manner the portion of the drain tube downstream of the separation filter has a preferential path for the air.

The fact of providing a separation filter, however, entails a considerable increase in the complexity and cost of said drainage system, since it is necessary to provide two drain tubes, one for the liquids and the other one for the air, as well as two connectors for connection to the collection chamber, which must indeed have a double coupling, while the drainage systems currently used commonly have a single coupling.

### SUMMARY

The aim of the present disclosure is to overcome the limitations of the background art described above, devising a thoracic drainage system that allows to obtain better effects than those obtainable with thoracic drainage systems of the known type, allowing the simultaneous and continuous evacuation of the fluids, i.e., of the air and of the liquids, that are present in the pleural space of the patient.

Within this aim, the present disclosure conceives a thoracic drainage system that facilitates a faster expansion of the lung by virtue of the simultaneous evacuation of the air and of the liquids, with the consequent reduction of the hospitalization times of the patient.

The present disclosure conceives a thoracic drainage system that minimizes the pain felt by the patient as well as the possibility of external contamination of said patient.

The present disclosure also devises a thoracic drainage system that allows continuous evacuation of the air in the collection chamber, allowing medical staff to visualize immediately any air leaks of the patient and providing medical staff with additional information on the air leaks of the patient.

The present disclosure also conceives a thoracic drainage system that allows to reduce the workload of the medical and nursing staff

The present disclosure further provides a thoracic drainage system that minimizes and possibly eliminates the unwanted occlusion events of the air and liquid drain tube.

The disclosure also devises a thoracic drainage system that substantially has standard components.

The disclosure further provides a thoracic drainage system the main components of which, such as for example the drainage catheter and the drain tube, can be provided by means of production methods that can be fully industrialized, in large numbers, at reduced costs, and in short times.

The present disclosure also provides a thoracic drainage system that is highly reliable, relatively simple to provide and at modest costs.

This aim, as well as these and other advantages which will become better apparent hereinafter, are achieved by providing a thoracic drainage system, comprising a drainage catheter adapted to be inserted in the pleural space of a patient in order to drain air and/or liquids that are present in said pleural space, and a drain tube, which is connected to said drainage catheter by means of a connector, which is adapted to make said air and/or said drained liquids flow out via said drainage catheter to a collection chamber, characterized in that said drainage catheter comprises, at its distal end, a plurality of longitudinally extended channels which are open radially toward the outside of said drainage catheter and are adapted to drain said liquids that are present in said pleural space, and, at its proximal end, at least one port for the drainage of said liquids, in which said open channels merge, said drainage catheter comprising a port for the drainage of said air that is present in said pleural space, said port for the drainage of said air comprising, at said distal end of said drainage catheter, a plurality of radial holes adapted to be crossed by said air that is present in said pleural space, said drain tube comprising, substantially along its entire longitudinal extension, a first outflow port which is connected, by means of said connector, to said at least one port for the drainage of said liquids and a second outflow port which is connected, by means of said connector, to said port for the drainage of said air.

### BRIEF DESCRIPTION OF THE DRAWINGS

Further characteristics and advantages of the disclosure will become better apparent from the description of a preferred but not exclusive embodiment of a thoracic drainage system according to the disclosure, illustrated by way of non-limiting example in the accompanying drawings, wherein:

FIG. 1 is a perspective view of a first embodiment of a thoracic drainage system according to the present disclosure;

FIG. 2 is a sectional view of the thoracic drainage system of FIG. 1, according to the present disclosure;

FIG. 3 is a perspective view of the distal end of a drainage catheter that belongs to the thoracic drainage system of FIG. 1, according to the present disclosure; FIG. 4 is a different perspective view of the distal end of the drainage catheter that belongs to the thoracic drainage system of FIG. 1, according to the present disclosure;

FIG. 5 is a further sectional perspective view of the distal end of the drainage catheter that belongs to the thoracic drainage system of FIG. 1, according to the present disclosure;

FIG. 6 is a perspective view of the proximal end of the drainage catheter that belongs to the thoracic drainage system of FIG. 1, according to the present disclosure;

FIG. 7 is a sectional exploded view of a connector that belongs to the thoracic drainage system of FIG. 1, according to the present disclosure;

FIG. 8 is a perspective view of the distal end of a drain tube which belongs to the thoracic drainage system of FIG. 1, according to the present disclosure;

FIG. 9 is a perspective view of a second embodiment of a thoracic drainage system according to the present disclosure;

FIG. 10 is a sectional view of the thoracic drainage system of FIG. 9, according to the present disclosure;

FIG. 11 is a perspective view of a drainage catheter that belongs to the thoracic drainage system of FIG. 9, according to the present disclosure;

FIG. 12 is a sectional exploded view of a connector that belongs to the thoracic drainage system of FIG. 9, according to the present disclosure;

FIG. 13 is a side view of a drainage catheter and of a drain tube that belong to a third embodiment of a thoracic drainage system, according to the present disclosure;

FIG. 14 is a sectional view of the drainage catheter shown in FIG. 13, taken along the plane XIV;

FIG. 15 is a sectional view of the drainage catheter shown in FIG. 13, taken along the plane XV; and

FIG. 16 is a sectional view of the drain tube shown in FIG. 13, taken along the plane XVI.

### DETAILED DESCRIPTION OF THE DRAWINGS

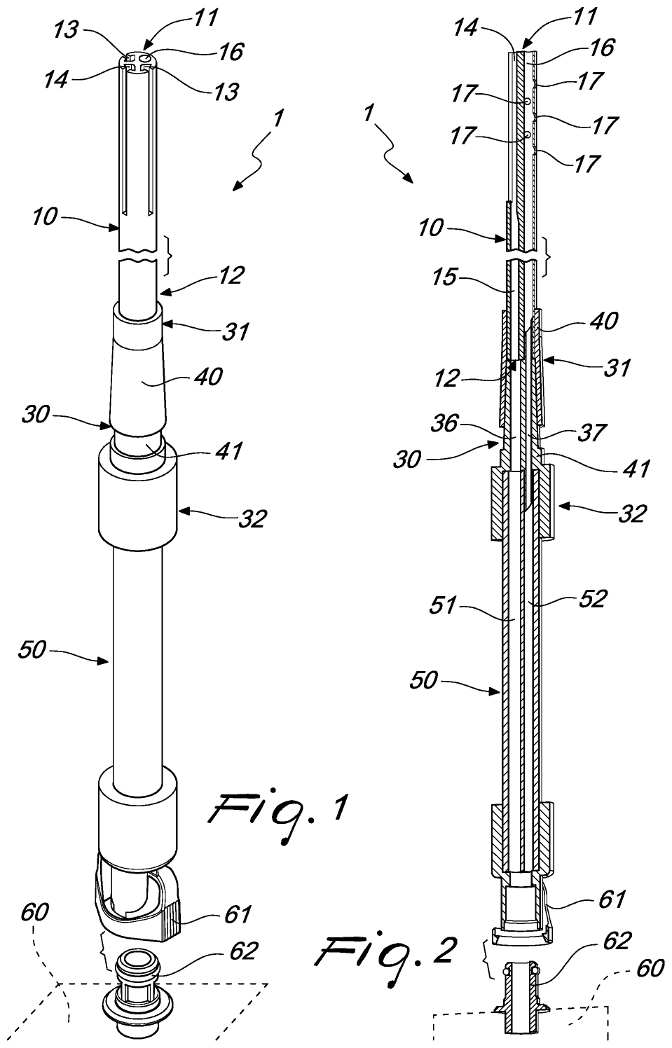

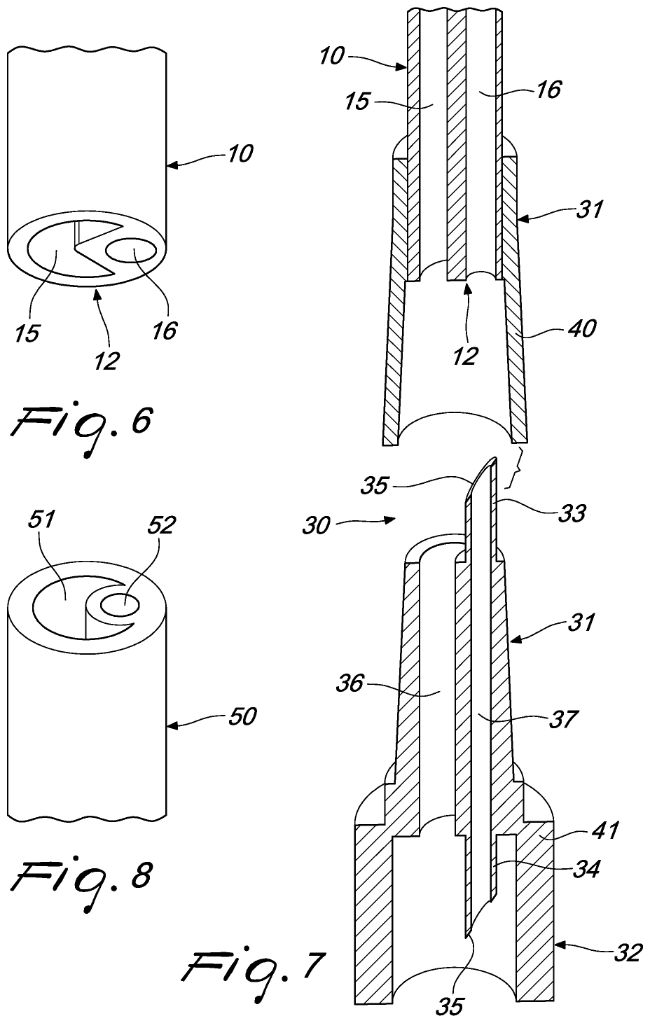

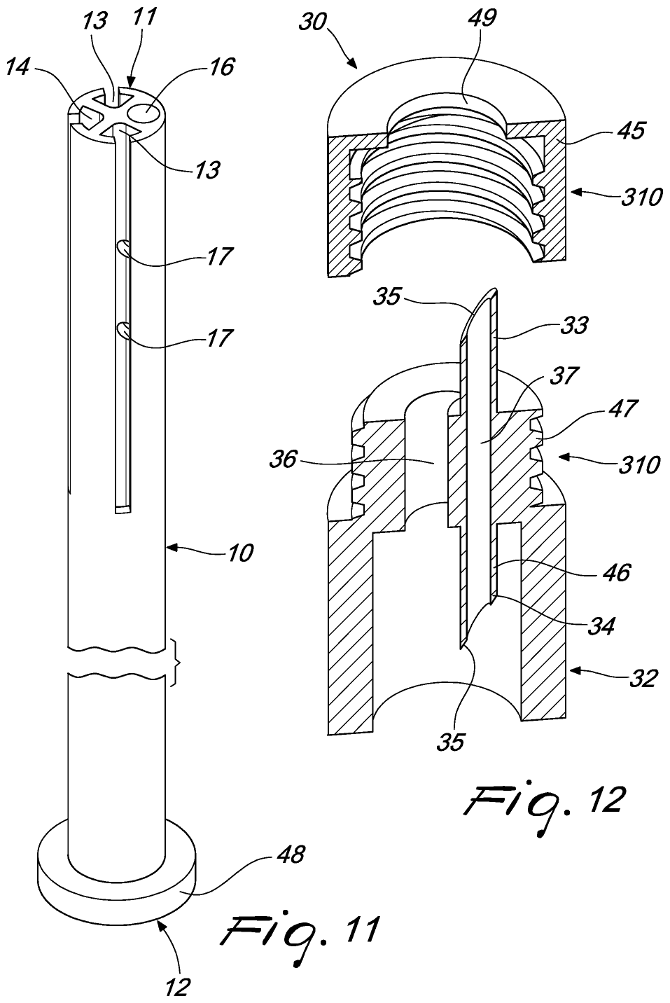

With reference to FIGS. 1-16, the thoracic drainage system, generally designated by the reference numeral 1, comprises a drainage catheter 10 adapted to be inserted in the pleural space of a patient in order to drain air and/or liquids that are present in the pleural space, and a outflow tube 50, which is connected to the drainage catheter 10 by means of a connector 30 which is adapted to make the drained air and/or liquids flow out via the drainage catheter 10 to a collection chamber 60.

According to the disclosure, the drainage catheter 10 comprises, at its distal end 11, a plurality of longitudinally extended channels 13, 14 which are open radially toward the outside of the drainage catheter 10 and are adapted to drain the liquids that are present in the pleural space, and, at its proximal end 12, at least one port 15, 150 for the drainage of said liquids, in which the open channels 13, 14 merge. The drainage catheter 10 furthermore comprises a port 16 for the drainage of the air that is present in the pleural space. The air drainage port 16 comprises, at the distal end 11 of the drainage catheter 10, a plurality of radial holes 17 adapted to be crossed by the air that is present in the pleural space.

Furthermore, according to the disclosure, the outflow tube 50 comprises, substantially along its entire longitudinal extension, a first outflow port 51, which is connected, by means of the connector 30, to the liquid drainage port 15, 150, and a second outflow port 52, which is connected, again by means of the connector 30, to the air drainage port 16.

In this manner, the air and the liquids that are present in the pleural space can be drained in separate channels along the extension of both the drainage catheter 10 and the outflow tube 50.

The term “proximal” is understood to reference that which is proximate to the base of the drainage system 1, i.e., essentially, to the collection chamber 60, while the term “distal” is understood to reference that which is furthest from the base of the drainage system 1, and therefore closest to the patient.

Advantageously, both the drainage catheter 10 and the outflow tube 50 can be obtained by means of processes for the extrusion of materials such as plastic, by virtue of the particular geometry of their transverse cross-sections.

Advantageously, the radial holes 17 for the passage of air inside the air drainage port 16 are arranged in the most distal portion of the drainage catheter 10, which is designed to reach, during use, the apical region of the pleural space, where the air to be drained accumulates more easily. In this manner it is possible to ensure a continuous drainage of the air that is present in the pleural space toward the collection chamber 60.

The open channels 13, 14 advantageously are extended along a greater length than the drainage catheter 10, affecting also portions of the catheter 10 located downstream, i.e., more proximally, with respect to where the radial holes 17 are arranged. In particular, the open channels 13, 14 are extended in the portion of the drainage catheter 10 that is designed to reach, during use, the basal region of the pleural space where the liquids to be drained instead tend to accumulate by gravity.

These open channels 13, 14 therefore allow the entry of the liquids that are present in the basal region of the pleural space and their drainage toward the liquid drainage port 15, 150 into which said open channels 13, 14 are conveyed.

In this manner it is possible to ensure continuous drainage also of the liquids that are present in the pleural space, toward the collection chamber 60.

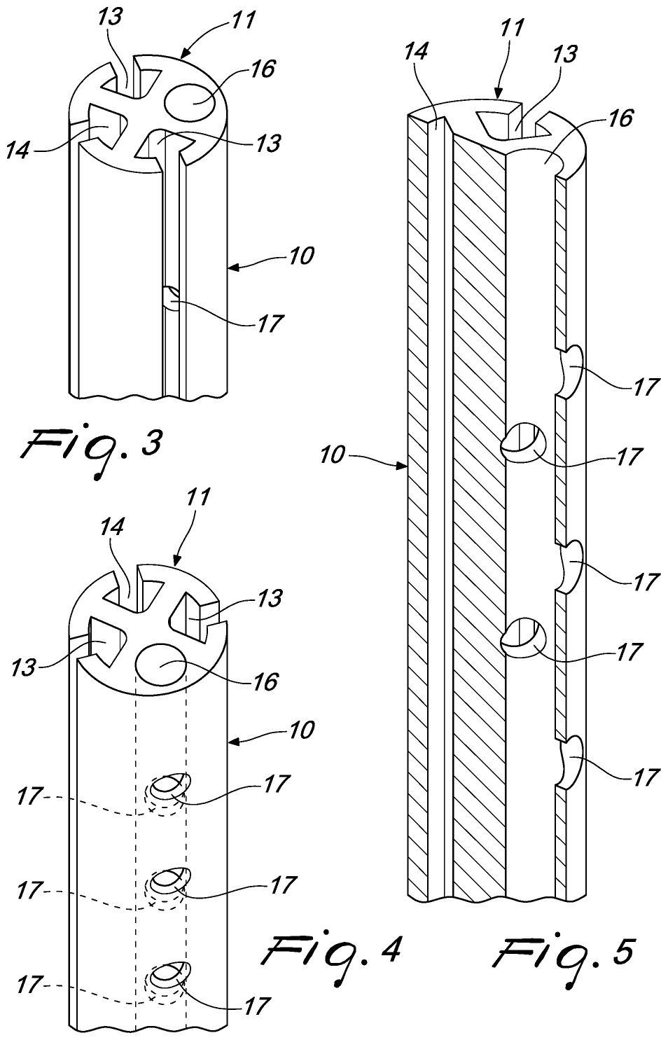

Advantageously, as shown in particular in FIGS. 3, 4 and 5, some radial holes 17 are directed radially toward the outside of the drainage catheter 10, while other radial holes 17 are directed toward at least one of the open channels 13, 14. For example, the air drainage port 16 can have radial holes 17 that are open toward the open channels 13 arranged laterally to said port 16.

In this manner, should the holes 17 that are directed toward the outside of the drainage catheter 10 become blocked, for example if they rest against the walls of the pleural space, air drainage would in any case be allowed through the holes that are directed toward the inside of the open channels 13.

Naturally, the number and/or breadth and/or arrangement of said radial holes 17 can vary in the various embodiments of the disclosure, while remaining within a value that does not affect negatively the very structure of the drainage catheter 10.

Advantageously, the drainage catheter 10 has, at the distal end 11, a substantially circular cross-section divided into at least two circular sectors. A first circular sector accommodates the air drainage port 16, while a second circular sector accommodates the open channels 13, 14.

In particular, as shown in FIGS. 3 and 4, the circular cross-section of the drainage catheter 10 can be divided into four circular sectors, of which one is occupied by the air drainage port 16 and the other three are occupied by the open channels 13, 14.

Advantageously, the drainage catheter 10 has, at the proximal end 12, a substantially circular cross-section divided into two circular sectors. One first circular sector accommodates the air drainage port 16, while a second circular sector accommodates the liquid drainage port 15, in which the open channels 13, 14 merge.

As shown in FIG. 6, at the proximal end 12 the drainage catheter 10 has a circular cross-section divided into two sectors, wherein the first sector, which accommodates the air drainage port 16, occupies approximately one quarter of the area of the cross-sectional circle, while the liquid drainage port 15 occupies the remaining part, i.e., approximately three quarters, of the area of the cross-sectional circle.

In an axial portion between the distal end 11 and the proximal end 12 of the drainage catheter 10, the open channels 13, 14 become closed channels, i.e., not open outward, and merge in the liquid drainage port 15.

As shown in FIGS. 1 to 12, which relate to the first and second embodiments of the drainage system 1, the air drainage port 16 is in an off-centered position with respect to the longitudinal axis of the drainage catheter 10.

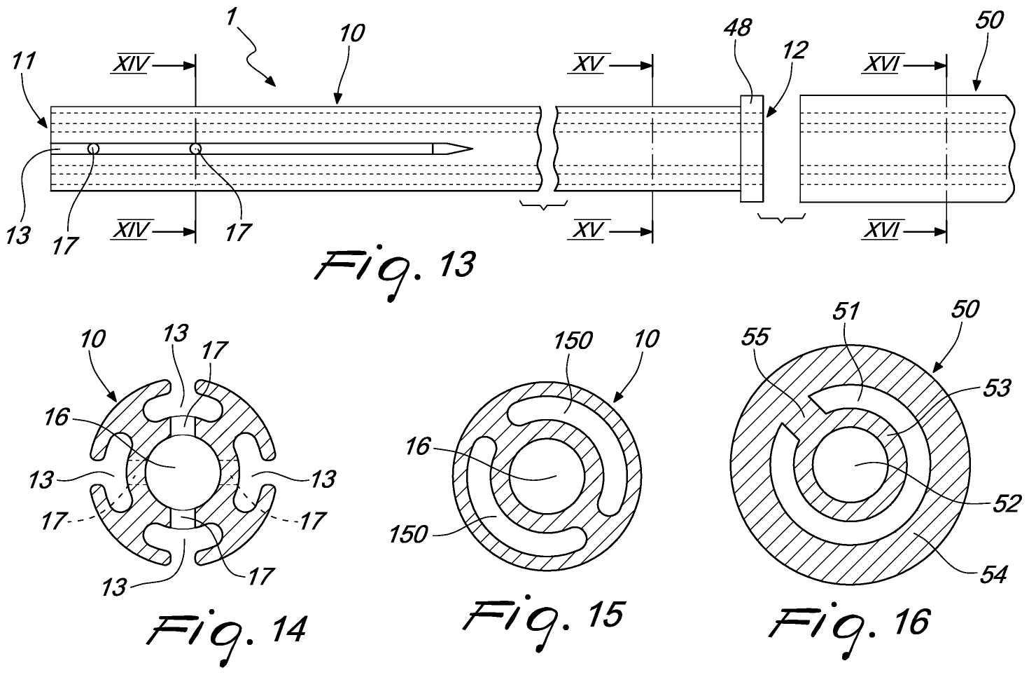

Advantageously, as shown in FIGS. 13 to 16, in a third embodiment of the drainage system 1 the drainage catheter 10 can have, at the distal end 11, a substantially circular cross-section, and the air drainage port 16 is advantageously arranged centrally with respect to said substantially circular cross-section, while the open channels 13, 14 are provided so as to be radially external with respect to the air drainage port 16.

For example, as shown in FIG. 14, the drainage catheter 10 has, radially more externally, four open channels 13, while the air drainage port 16 is arranged at the center.

At the proximal end 12 it is possible to provide a pair of liquid drainage ports 150 which have, as shown in FIG. 15, a substantially semiannular cross-section and are arranged radially external with respect to the air drainage port 16.

In this third embodiment of the drainage system 1, in the drainage catheter 10, between the distal end 11 and the proximal end 12, the open channels 13 become closed and merge in pairs in the two liquid drainage ports 150.

Advantageously, furthermore, in this third embodiment of the drainage system 1 the outflow tube 50 comprises an internal tube 53, which defines the second outflow port 52, and an external tube 54. Between the external tube 54 and the internal tube 53 there is an interspace that constitutes the first outflow port 51. Advantageously, the internal tube 53 is arranged substantially at the center of the external tube 54 and is associated therewith by means of a rib 55 extended longitudinally along substantially all of the outflow tube 50.

Advantageously, the connector 30 is provided with a first portion 31, 310, which can be associated with the drainage catheter 10 by interference or screwing, and a second portion 32, which is arranged opposite the first portion 31 and can be associated with the outflow tube 50 by interference and/or adhesive bonding.

Furthermore, the connector 30 advantageously comprises a first connecting duct 36 which is arranged to connect the liquid drainage port 15 to the first outflow port 51, and a second connecting duct 37 that is arranged to connect the air drainage port 16 to the second outflow port 52. Furthermore, the second connecting duct 37 comprises, at both of its ends, two cylindrical walls 33, 34 which protrude axially and are arranged to engage respectively in the air drainage port 16 and in the second outflow port 52.

Advantageously, the cylindrical walls 33, 34 comprise a guiding bevel 35 for engagement within the air drainage port 16 and within the second outflow port 52.

In a first embodiment of the drainage system 1, shown in FIGS. 1 to 8, the connector 30 comprises a sleeve 40 and a connecting body 41. The sleeve 40 is configured to retain together, by interference, the proximal end 12 of the drainage catheter 10 with the connecting body 41. The connecting body 41 is instead associated, by interference and/or adhesive bonding, with the outflow tube 50.

Preferably, the connector 30 is joined to the outflow tube 50 by means of chemical bonding methods.

The cylindrical walls 33, 34 for coupling in the air drainage port 16 and in the second outflow port 52 are advantageously provided by the connecting body 41.

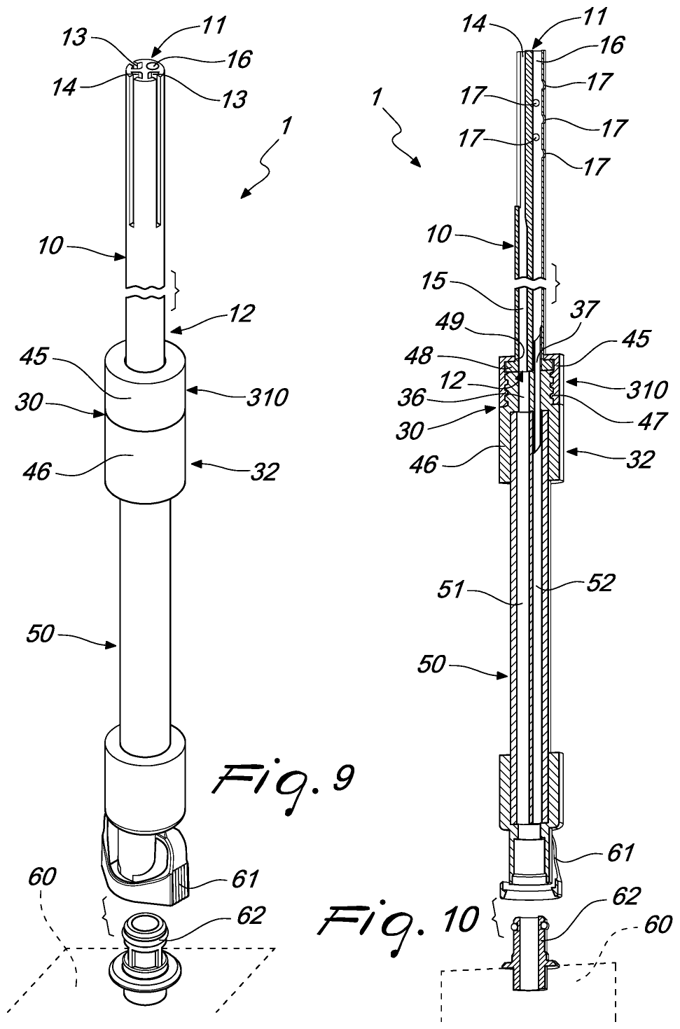

In a second embodiment of the drainage system 1, shown in FIGS. 9 to 12, the connector 30 comprises a threaded ring 45 and a connecting body 46, which comprises a threaded head 47.

The connecting body 46 is advantageously associated by interference with the outflow tube 50.

The drainage catheter 10 comprises, at its proximal end 12, a collar 48 which protrudes radially and is configured to be retained between the threaded ring 45 and the connecting body 46.

In particular, the threaded ring 45 is provided with a through hole 49 which has such a diameter that it can be crossed by the body of the drainage catheter 10 but not by the corresponding terminal collar 48.

The fastening of the threaded ring 45 on the threaded head 47 of the connecting body 46 allows to block the collar 48 and therefore the drainage catheter 10.

A quick coupling connector 61 is advantageously joined, for example by chemical bonding, in the proximal part of the outflow tube 50 and is configured to engage a corresponding connector 62 that is present on the collection chamber 60.

The quick coupling connector 61 is advantageously a connector of the standard type, which can be associated, at the operator's discretion, with collection systems of various kinds, such as for example mechanical collection systems, water-based systems, gravity systems, with centralized suction, with suction by means of a vacuum unit, or other collection systems provided with systems for recording intrapleural pressure data.

In practice it has been found that the disclosure achieves fully the intended aims and advantages. In particular, it has been shown that the thoracic drainage system thus conceived allows to overcome the quality limitations of the background art, since it allows the simultaneous and continuous evacuation of the fluids, i.e., of the air and of the liquids, that are present in the thoracic cavity of the patient.

Another advantage of the thoracic drainage system according to the disclosure resides in that its main components, i.e., the drainage catheter, the drain tube and the corresponding connector, can be provided by means of methods that can be easily industrialized, such as for example extrusion and/or molding, without requiring complicated subsequent processes. In particular, the provision of the extruded components, i.e., of the drainage catheter and of the drain tube, does not require particular adhesive bonding or welding steps.

A further advantage of the thoracic drainage system according to the disclosure resides in that it minimizes and even eliminates the harmful occlusion events of the air and/or liquid evacuation channels, always ensuring a free path for the drainage of the air from the pleural space to the collection chamber.

Another advantage of the thoracic drainage system according to the disclosure resides in that it can be interfaced with collection chambers of the known and standard type and does not require the adoption of particular separation filters.

In particular, the thoracic drainage system according to the disclosure allows to record the data of the intrapleural pressures without variations that might be caused by the application of intermediate separation filters.

Another advantage of the thoracic drainage system according to the disclosure resides in that it facilitates a more rapid expansion of the lung, by virtue of the simultaneous evacuation of the air and of the liquids, with consequent reduction of the hospitalization times of the patient.

A further advantage of the thoracic drainage system according to the disclosure resides in that it facilitates its management, and more generally the management of the thoracic drainage system, and minimizes the intensity of the pain felt by the patient.

Furthermore, the drainage catheter according to the disclosure can be provided to any drainage system, be it with one or more collection chambers, be it provided or not with valves for the application and adjustment of suction, be it equipped or not with a vacuum unit or connected to the centralized hospital vacuum system. Said coaxial drainage catheter performs its function regardless of the collection system to which it is connected.

The disclosure thus conceived is susceptible of numerous modifications and variations, all of which are within the scope of the appended claims; by way of nonlimiting example, the person skilled in the art understands without effort that it is also possible to provide a mechanism or a valve for interrupting the flow of fluids in input or for interrupting the flow of liquids and air in output. Furthermore, all the details may be replaced with other technically equivalent elements.

In practice, the materials used, as well as the contingent shapes and dimensions, may be any according to the requirements and the state of the art.

To conclude, the scope of the protection of the claims must not be limited by the illustrations or preferred embodiments shown in the description by way of example, but rather the claims must comprise all the characteristics of patentable novelty that reside in the present disclosure, including all the characteristics that would be treated as equivalents by the person skilled in the art.

The disclosures in Italian Patent Application No. 102016000124793 (UA2016A008934) from which this application claims priority are incorporated herein by reference.

1-10 (canceled)

11. A thoracic drainage system comprising:
a drainage catheter adapted to be inserted in the pleural space of a patient in order to drain air and/or liquids that are present in said pleural space, and
a drain tube connected to said drainage catheter by means of a connector adapted to make said air and/or said drained liquids flow out via said drainage catheter to a collection chamber, wherein said drainage catheter comprises, at its distal end, a plurality of longitudinally extended channels which are open radially toward the outside of said drainage catheter and are adapted to drain said liquids disposed in said pleural space, and, at its proximal end, at least one port for the drainage of said liquids, in which said open channels merge, said drainage catheter comprising a port for the drainage of said air disposed in said pleural space, said port for the drainage of said air comprising, at said distal end of said drainage catheter, a plurality of radial holes adapted to be crossed by said air disposed in said pleural space, said drain tube comprising, substantially along its entire longitudinal extension, a first outflow port which is connected, by means of said connector, to said at least one port for the drainage of said liquids and a second outflow port which is connected, by means of said connector, to said port for the drainage of said air.

12. The thoracic drainage system according to claim 11, wherein at least some of said radial holes of said port for the drainage of said air are directed toward at least one of said open channels.

13. The thoracic drainage system according to claim 11, wherein said drainage catheter has, at said distal end, a substantially circular cross-section divided into two circular sectors, a first circular sector accommodating said port for the drainage of said air, a second circular sector accommodating said plurality of open channels.

14. The thoracic drainage system according to claim 11, wherein said drainage catheter has, at said proximal end, a substantially circular cross-section that is divided into a first circular sector accommodating said port for the drainage of said air and a second circular sector accommodating said port for the drainage of said liquids into which said open channels merge.

15. The thoracic drainage system according to claim 11, wherein said drainage catheter has, at said distal end, a substantially circular cross-section, said port for the drainage of said air being arranged centrally with respect to said substantially circular cross-section, said plurality of open channels being provided so as to be radially external to said port for the drainage of said air.

16. The thoracic drainage system according to claim 11, wherein said drainage catheter comprises, at said proximal end, a pair of ports for the drainage of said liquids which have a substantially semiannular cross-section and are arranged so as to be radially external with respect to said port for the drainage of said air.

17. The thoracic drainage system according to claim 11, wherein said drainage catheter and/or said drain tube can be obtained by means of processes for the extrusion of materials such as plastic.

18. The thoracic drainage system according to claim 11, wherein said connector is provided with a first portion, which can be associated with said drainage catheter by interference or screw coupling, and a second portion, which is opposite with respect to said first portion and can be associated with said drain tube by interference.

19. The thoracic drainage system according to claim 11, wherein said connector comprises a first connecting duct that is arranged to connect said at least one port for the drainage of said liquids to said first outflow port and a second connecting duct that is arranged to connect said port for the drainage of said air to said second outflow port, said second connecting duct comprising, at both of its ends, two cylindrical walls which protrude axially and are arranged to engage respectively in said port for the drainage of said air and in said second outflow port.

20. The thoracic drainage system according to claim 19, wherein said cylindrical walls comprise a guiding bevel for coupling in said port for the drainage of said air and in said second outflow port.
